(19) United States
(12) Patent Application Publication
(10) Pub. No.: US 2017/0224522 A1
(43) Pub. Date: Aug. 10, 2017

(54) VACUUM THERAPY APPARATUS

(71) Applicant: Global Health & Innovation, Miami, FL (US)

(72) Inventor: Norman Ivan IRIAS, Miami, FL (US)

(73) Assignee: Global Health & Innovation, Miami, FL (US)

(21) Appl. No.: 15/016,625

(22) Filed: Feb. 5, 2016

(51) Int. Cl. A61F 5/41; A61H 19/00

(52) U.S. Cl. CPC A61F 5/41; A61H 19/32; A61F 2005/412

(57) ABSTRACT

A vacuum therapy apparatus includes a vacuum chamber configured to receive an organ to be treated, a vacuum pump coupled to the vacuum chamber and configured to draw air from the vacuum chamber so that blood is drawn into the organ, a controller configured to cause the vacuum pump to undergo an operating cycle in which the organ is changed from a flaccid to an erectile state and then back to an flaccid state, causing tissues of the organ to expand and contract, and a memory provided on a detachable part of the apparatus and configured to store information regarding at least one operating cycle under which the apparatus has operated.

### TECHNICAL FIELD

The present invention relates to a compete automatic vacuum therapy apparatus for a patient configured for recording of patient usage report data and transfer thereof to the physician.

### BACKGROUND DISCUSSION

Devices which use a vacuum to draw blood into a patient's penis have several proven medical uses, including treatment of erectile dysfunction, penile rehabilitation after treatment for prostate cancer, reduction in penile curvature caused by Peyronie's disease, and pre-operative and post-operative treatment of penile implantation surgery. However, existing vacuum therapy devices can be problematic in terms of proper usage and tracking of such usage. Accordingly, a need exists for a vacuum therapy device in which the patient can easily use the device for a treatment regimen tailored to the patient's needs, and in which information regarding such treatments can be easily gathered and transferred to a caregiver as needed.

### SUMMARY

The present application discloses a vacuum therapy apparatus including a vacuum chamber configured to receive an organ to be treated, a vacuum pump coupled to the vacuum chamber and configured to draw air from the vacuum chamber so that blood is drawn into the organ, a controller configured to cause the vacuum pump to undergo an operating cycle in which the organ is changed from a flaccid to an erectile state and then back to an flaccid state, causing tissues of the organ to expand and contract, and a memory provided on a detachable part of the apparatus and configured to store information regarding at least one operating cycle under which the apparatus has operated.

In an embodiment, a communication interface provided on the detachable part is configured to transmit the information to a separate device, and the controller stores at least one operating cycle program which, when selected by a user via on operation interface of the apparatus, causes the controller to execute the operating cycle according to the operating cycle program.

### BRIEF DESCRIPTION OF THE DRAWINGS

FIG. 1 is a perspective view of a vacuum therapy apparatus.

FIG. 2 is an exploded view of a top portion of the vacuum therapy apparatus.

FIG. 3 is an exploded view of a front section of the top portion of the vacuum therapy apparatus.

FIG. 4 is an exploded view of a base section of the top portion of the vacuum therapy apparatus.

FIG. 5 is an exploded view of a back section of the top portion of the vacuum therapy apparatus and elements attached thereto.

FIG. 6 is an exploded view of a detachable section of the top portion of the vacuum therapy apparatus and elements attached thereto.

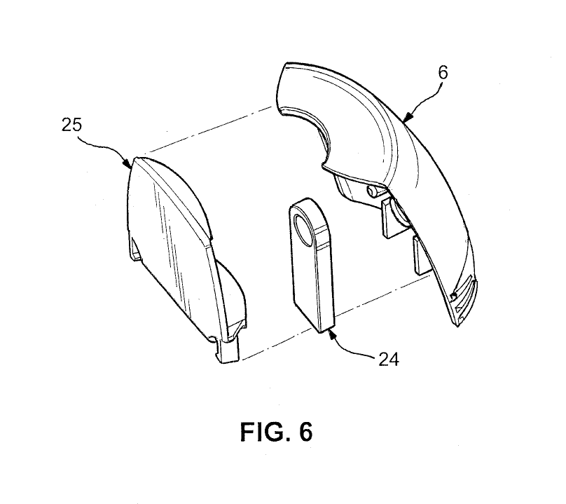

### DETAILED DESCRIPTION

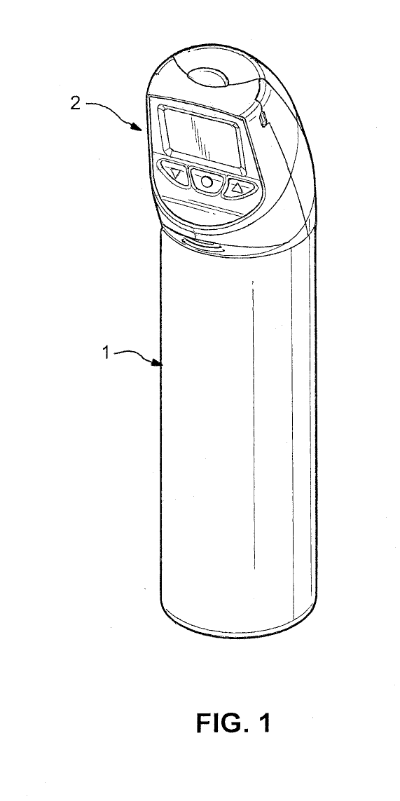

A vacuum therapy apparatus illustrated in FIG. 1 includes a vacuum tube 1 and a top portion 2. The vacuum tube 1 defines to a vacuum chamber configured to receive an organ to be treated, i.e., is configured for insertion of the patient's penis, and is, for example, a clear plastic cylinder.

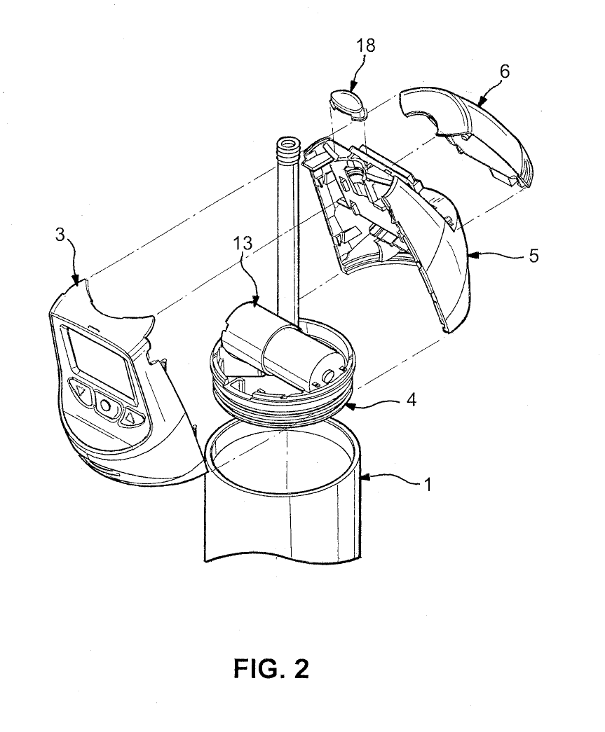

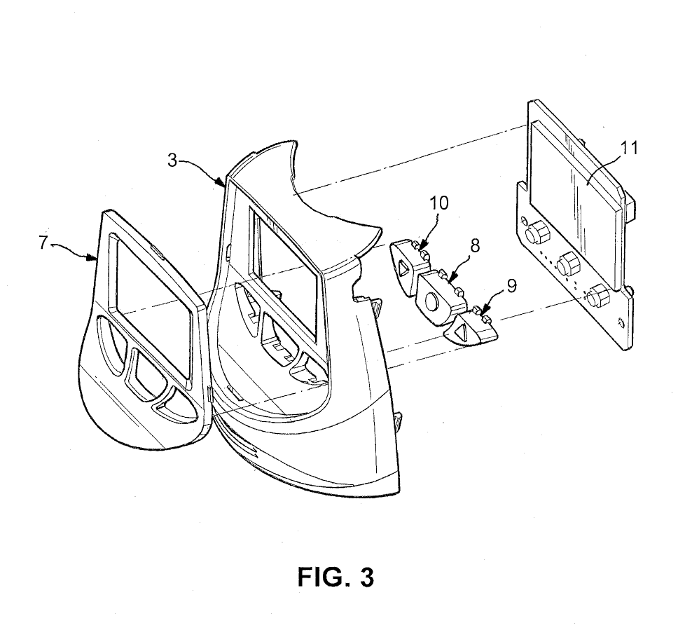

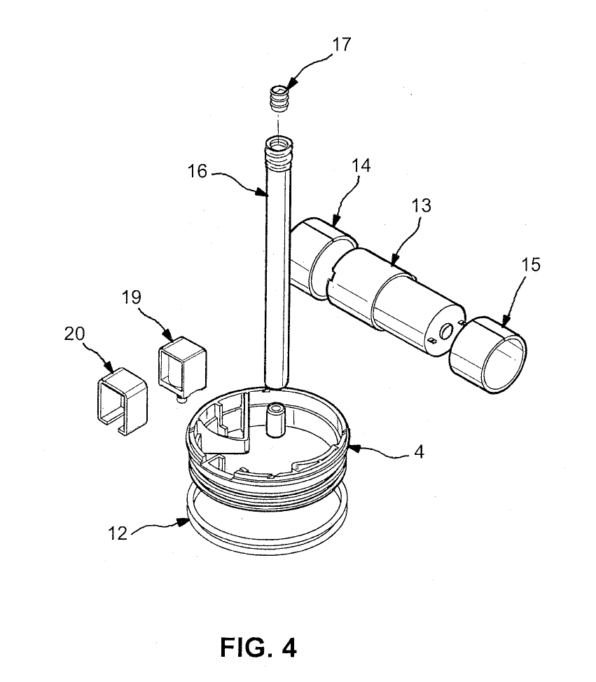

As illustrated in FIG. 2, the top portion 2 includes a front case part 3, a base part 4, a back case part 5, and a detachable part 6. The front case part 3 is connected to the base part 4, for example, by a snap fit. The base part 4 is attached to the vacuum tube 1, such as by snap-fitting, with an o-ring 12 (see FIG. 4) being provided to seal the interface between the vacuum tube 1 and the base part 4. The back case part 5 is connected to the front case part 3 and the base part 4, for example, by snap fitting. As illustrated in FIG. 3, a bezel 7 is attached at an outside of the front case part 3, and a home button 8, a forward button 9, a back button 10, and a screen 11 are attached at an inside of the front case part 3 and operatively connected to a USB Board 21 which is described in detail below.

In particular, the home button 8, when pressed while the device is off or asleep, turns on or wakes up the device (if the device has entered a sleep mode due, for example, to inactivity. When held down for several seconds, the home button 8 turns off the device. When the device is turned on and awake, names of various operating modes, which are described in detail below, are displayed on the screen 11, and the buttons 9 and 10 can be used to scroll through and selectively highlight the operating mode names. When a particular operating mode name is selected and the home button 8 is pressed, the processor of the USB board 21 will run the program which corresponds to the selected operating mode. The details of such operation are described below.

The base part 4 sealingly closes an end of the vacuum tube 1 opposite the end in which the penis is to be inserted. As illustrated in FIG. 4, an electrical air pump 13 is mounted to the base part 4 by, for example, foam tape pieces 14 and 15, on a side of the base part 4 opposite the vacuum tube 1. The air pump 13 is configured to apply vacuum to the vacuum tube 1 through an opening in the base part 4. Furthermore, a release valve 19 which allows air to exit throught a vent of the pump 13 is kept in position via foam tape 20. The air pump 13 therefore corresponds to a vacuum pump coupled to a vacuum chamber and configured to draw air from the vacuum chamber so that blood is drawn into the organ.

Connected to a second passageway in the base part 4 is a relief tube 16 which further includes, at its free end, a release actuator 17 which can be actuated by a user via a push button 18, as discussed in detail below. When the release actuator 17 is actuated, the release valve 19 is opened thereby releasing vacuum which has built up in the vacuum tube 1 by allowing outside air to enter the vacuum tube 1. This allows the user to release the pressure in the device prior to the completion of an operating cycle if, for example, the pressure becomes unbearable to the user or the user needs to attend to an unexpected emergency. As the vacuum within the tube is likely to cause the user's skin to adhere to the tube, the release valve 19 is needed to break the suction and allow the user to disengage from the device.

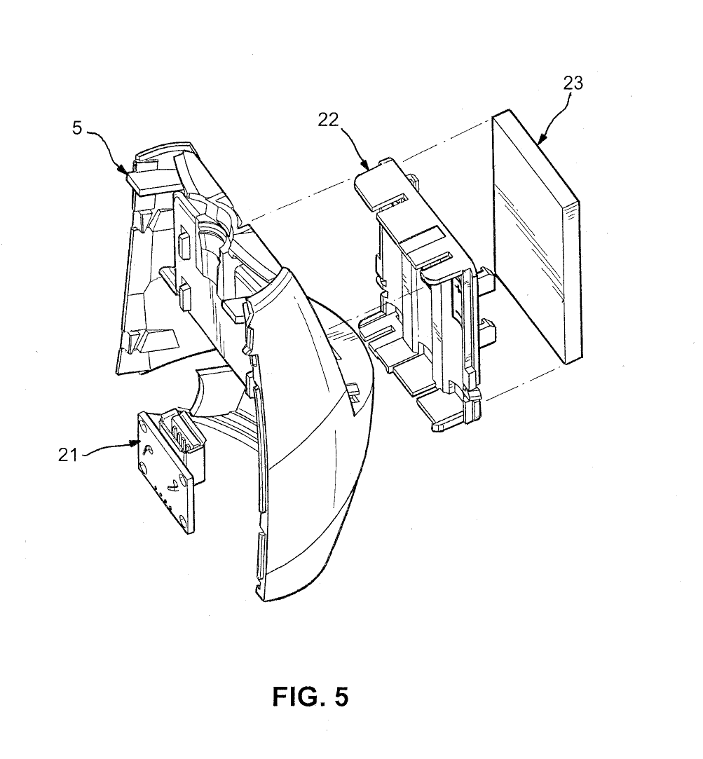

As illustrated in FIG. 5, the back case part 5 has a USB board 21 and a battery tray 22 attached thereto, such as by snap fitting. The USB board 21 includes a processor, a memory, and a USB port. A battery 23 is provided on the battery tray 22, and the battery tray 22 is operatively connected to the USB board 21 such that the battery 23 can power the USB board 21. The processor of the USB board is operatively connected to the pump 13, the release valve 19, and a pressure sensor which indicates the pressure in the vacuum tube 1, and is configured to run one or more programs saved in memory to actuate the pump 13 and the release valve 19 according to predetermined steps. The USB board 21 corresponds to a controller configured to cause the vacuum pump to undergo an operating cycle in which the organ is changed from a flaccid to an erectile state and then back to an flaccid state, causing tissues of the organ to expand and contract.

The treatment programs saved in the memory will typical follow the following configuration, with the particular pressures and durations varying according to the type of therapy provided by the program:

1-30 min a day therapy at a pressure of 1-50 Kpa
1—Pump 0.1-20 seconds
2—Pause 0.1-20 seconds
3—Pump 0.1-20 seconds
4—Pause 0.1-20 seconds
5—Pump 0.1-20 seconds
6—Pause 0.1-60 seconds and release
   Repeat steps 1 to 6 for 1-30 min.

The pump and pause steps are initiated by automatic actuation and de-actuation of the air pump 13, while the release step is initiated by automatic opening of the release valve 19. The controller of the device therefore stores at least one operating cycle program which, when selected by a user via on operation interface of the apparatus, causes the controller to execute the operating cycle according to the operating cycle program. While the operating cycle corresponding to the selected mode is running on the device, the screen displays various parameters such as time remaining and current negative pressure. Furthermore, a plurality of operating programs corresponding to different therapies, such as a therapy for treatment of erectile dysfunction, a therapy for penile rehabilitation after treatment for prostate cancer, a therapy for reduction in penile curvature caused by Peyronie's disease, and a therapy for pre-operative and post-operative treatment of penile implantation surgery, can be pre-loaded into the memory of the USB board 21. The device can further be configured such that, by changing the program corresponding to a therapy stored in the memory of the USB board 21, a therapy can be modified by a user as needed to best fit a patient's needs.

The detachable part 6 of the top portion 2 is connected to the back case part 5, for example, by an easily detachable snap fitting, and has a flash drive 24 secured thereto by a cover 25. The detachable part 6 is configured such that, when it is connected to the back case part 5, the flash drive 24 is connected to the USB port of the USB board 21. After each operating cycle is run, information regarding the operating cycle is saved onto the flash drive 24.

When the detachable part 6 is removed from the apparatus and the cover 25 removed from the back case part 5, the flash drive 24 can be removed from the detachable part 6 and attached to a separate device, such as the computer of a caregiver. The information on the flash drive 24 can then be accessed, transferred, and cleared from the flash drive 24 as necessary. The flash drive 24 therefore corresponds to a memory provided on the detachable part and configured to store information regarding at least one operating cycle under which the apparatus has operated, and a communication interface provided on the detachable part and configured to transmit the information to a separate device, such as a computer.

The detailed description above describes a vacuum therapy apparatus. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A vacuum therapy apparatus comprising:
a vacuum chamber configured to receive an organ to be treated;
a vacuum pump coupled to the vacuum chamber and configured to draw air from the vacuum chamber so that blood is drawn into the organ;
a controller configured to cause the vacuum pump to undergo an operating cycle in which the organ is changed from a flaccid to an erectile state and then back to an flaccid state, causing tissues of the organ to expand and contract; and
a memory provided on a detachable part of the apparatus and configured to store information regarding at least one operating cycle under which the apparatus has operated.

2. The vacuum therapy apparatus of claim 1, further comprising:
a communication interface configured to transmit the information to a separate device.

3. The vacuum therapy apparatus of claim 2, wherein the communication interface is provided on the detachable part.

4. The vacuum therapy apparatus of claim 1, wherein the controller stores at least one operating cycle program which, when selected by a user via on operation interface of the apparatus, causes the controller to execute the operating cycle according to the operating cycle program.
